(19) United States
(12) Patent Application Publication
(10) Pub. No.: US 2026/0192135 A1
(43) Pub. Date: Jul. 9, 2026

(54) Implantable Nano-Ultrasonic Cavitation Therapeutic Particle

(71) Applicant: Zhijun Peng, Foshan (CN)

(72) Inventor: Zhijun Peng, Foshan (CN)

(21) Appl. No.: 19/021,193

(22) Filed: Jan. 15, 2025

(30) Foreign Application Priority Data
Jan. 8, 2025 (CN) 2025100298822

(51) Int. Cl. A61N 7/00; A61B 17/22

(52) U.S. Cl. CPC A61N 7/00; A61B 2017/22008; A61B 17/2202; A61N 2007/0047

(57) ABSTRACT

An implantable nano-ultrasonic cavitation therapeutic particle, comprising a first hemispherical shell, a central shell, and a second hemispherical shell, wherein the first and second hemispherical shells are connected via the central shell, forming a complete microspherical particle; the central shell houses a micro-ultrasonic vibration assembly having a sliding block with an integrated permanent magnet, buffer springs, and a driving coil configured to interact with the permanent magnet to generate a magnetic driving force; the first hemispherical shell incorporates a drive control assembly, the drive control assembly including a circuit board with an integrated wireless communication module and a wireless charging receiving coil electrically coupled to the circuit board, the driving coil being electrically connected to the circuit board and generating a magnetic driving force causing the sliding block to reciprocate at high frequency; the second hemispherical shell further incorporates a first battery electrically coupled to the circuit board.

### TECHNICAL FIELD

The present invention relates to the field of ultrasonic therapeutic devices, and more particularly, to an implantable nano-ultrasonic cavitation therapeutic particle.

### BACKGROUND ART

Ultrasonic therapeutic devices use ultrasonic waves as a therapeutic source, focusing external ultrasonic waves on diseased tissues inside the body, causing the temperature of the diseased tissues to rise rapidly. Under the mechanical effects, thermal penetration, and cavitation, biological effects are produced, thereby killing and ablating the diseased tissue cells, preventing the spread of the diseased tissue cells, and achieving the therapeutic purpose. At the same time, the ultrasonic cavitation effect can also promote blood circulation, accelerate metabolism, and enable healthy cell tissues to regenerate, thereby achieving the therapeutic purpose for diseased tissues.

In existing ultrasonic therapeutic devices, as disclosed in patent documents published by the China National Intellectual Property Administration, a technical solution named “A Real-time Positioning Low-Energy Ultrasonic Therapeutic Device” was disclosed in Patent Application No. 202010504927.4, Publication No. CN111544782A. This solution consists of two parts: a therapeutic device host and an ultrasonic probe. The therapeutic device host is composed of a supporting device, a control panel, an image display screen, and an ultrasonic wave generating device. The ultrasonic probe is connected to the ultrasonic wave generating device. During treatment, the doctor applies the ultrasonic probe to the surface of the human body, using the ultrasonic probe to output ultrasonic energy to the diseased area of the human body for ultrasonic treatment. In the actual clinical practice, it has been found that this type of ultrasonic therapeutic device still has some shortcomings: First, since this type of ultrasonic therapeutic device is an external ultrasonic therapeutic device, the ultrasonic probe needs to use a relatively high power so that the ultrasonic energy can reach the diseased area inside the body from the external skin and achieve ultrasonic treatment of the diseased area. However, a higher-power ultrasonic probe will cause a larger ultrasonic radiation area, which will cause certain damage to the skin tissue on the surface of the human body, and the skin is prone to intermittent colic and laceration-like minor injuries. At the same time, for the diseased area inside the body, the ultrasonic energy is attenuated after passing through the skin and muscles, and when it reaches the diseased area, the therapeutic effect of the ultrasonic wave is weakened, which increases the treatment time and frequency, increases the treatment cost, and increases the number of times the skin tissue on the surface of the human body is damaged, thereby increasing the suffering of the patient. Second, since this type of ultrasonic therapeutic device is professional medical equipment, it requires professional doctors to operate. Patients can only follow the doctor's instructions and make repeated trips to the hospital for treatment, which requires a lot of time and money. Therefore, based on the shortcomings and drawbacks of existing ultrasonic therapeutic devices, the applicant believes that there is a great need for a new form of ultrasonic therapeutic device to replace and change the existing ultrasonic therapeutic devices, helping to reduce the trauma and suffering of patients and improve the therapeutic effect.

### SUMMARY OF THE INVENTION

The purpose of the present invention is to solve the above problems and deficiencies, and to provide an implantable nano-ultrasonic cavitation therapeutic particle, which is convenient to implant into the diseased area inside the human body. The ultrasonic cavitation effect directly acts on the diseased area, ensuring that the diseased tissue cells are killed without harming the skin on the surface of the human body, reducing the number of trips to the hospital for patients, and saving time and money.

The technical solution of the present invention is implemented as follows: an implantable nano-ultrasonic cavitation therapeutic particle, including a first hemispherical shell, a central shell, and a second hemispherical shell, wherein the first and second hemispherical shells are connected via the central shell, forming a complete microspherical particle; the central shell houses a micro-ultrasonic vibration assembly having a sliding block with an integrated permanent magnet, buffer springs positioned at opposing ends of the sliding block, and a driving coil configured to interact with the permanent magnet to generate a magnetic driving force; the first hemispherical shell incorporates a drive control assembly, the drive control assembly including a circuit board with an integrated wireless communication module and a wireless charging receiving coil electrically coupled to the circuit board, the driving coil being electrically connected to the circuit board and generating a magnetic driving force causing the sliding block to reciprocate at high frequency; the second hemispherical shell further incorporates a first battery electrically coupled to the circuit board.

Preferably, the first hemispherical shell and the central shell further include a first interlocking positioning pin and a first positioning hole, and the second hemispherical shell and the central shell further include a second interlocking positioning pin and a second positioning hole.

Preferably, the first hemispherical shell and the central shell are further provided with a first sealing ring, and the second hemispherical shell and the central shell are further provided with a second sealing ring.

Preferably, the first hemispherical shell or the second hemispherical shell further incorporates a camera module or a sensor module electrically coupled to the circuit board.

Preferably, the outer surface of the microsphere particle is further provided with a plurality of flow guiding grooves arranged in an intersecting pattern.

Preferably, the present invention further comprises an external controller; wherein the external controller includes a housing, a main control circuit disposed within the housing and configured for bidirectional control signal communication with the wireless communication module, and an operation display assembly mounted on the housing, the operation display assembly being electrically coupled to the main control circuit.

Preferably, the operation display assembly comprises a display screen and control buttons, the display screen and control buttons being each electrically coupled to the main control circuit.

Preferably, the housing further includes a wireless charging transmitting coil electrically coupled to the main control circuit, wherein the wireless charging transmitting coil is configured to transmit an electromagnetic field to the wireless charging receiving coil, and wherein the wireless charging receiving coil is configured to convert the received electromagnetic field into a current for charging the first battery.

Preferably, the housing further incorporates a second battery electrically coupled to the main control circuit, and wherein the housing is further provided with a charging interface electrically connected to the second battery.

Preferably, the housing further incorporates a DC electromagnet electrically coupled to the main control circuit, and wherein the surface of the microsphere particle is provided with a micro-magnet configured for magnetic interaction with the DC electromagnet; the surface of the microsphere particle further includes a recessed mounting slot, and the micro-magnet is embedded within the mounting slot.

Due to its nano-scale size and spherical shape, the therapeutic particle can be more easily implanted into the diseased area inside the human body. The cavitation effect generated by ultrasonic waves is concentrated and directly acts on the diseased area, ensuring that the diseased tissue cells are killed, effectively improving the effect of ultrasonic treatment, and not causing trauma to the skin on the surface of the human body. It can effectively avoid the irreversible damage to human organs caused by excessive medication and excessive traumatic surgery in traditional medical methods. At the same time, because the therapeutic particle is implanted in the human body and its volume is extremely small, the patient does not feel any foreign body sensation. It can perform timed treatment inside the human body without affecting the patient's daily life and work. The therapeutic particle has the function of wireless charging and wireless operation control. Once implanted, it can be used in the human body for a long time. Patients can use the wireless communication module to connect to a smartphone and use software to receive ultrasonic treatment at regular intervals according to their needs, without frequent visits to the hospital, greatly reducing the number of trips to the hospital, saving time and money. In addition, the nano-scale spherical design of this solution allows it to be implanted into the diseased area of the human body through surgery or injection, and the surgical wound is extremely small, so it does not affect the patient's life and work after the operation, greatly reducing the patient's pain. The product of this solution can be implanted into human tumor sites, inside blood vessels or the interlayer walls of blood vessels, the gallbladder, and other diseased human organs for treatment.

### DESCRIPTION OF DRAWINGS

FIG. 1 is a schematic diagram of the three-dimensional structure of the microsphere particle of the present invention.

FIG. 2 is a schematic diagram of a partially exploded structure of the microsphere particle of the present invention.

FIG. 3 is a first schematic diagram of an exploded structure of the microsphere particle of the present invention.

FIG. 4 is a second schematic diagram of an exploded structure of the microsphere particle of the present invention.

FIG. 5 is a schematic diagram of an exploded structure of the central shell of the present invention.

FIG. 6 is a schematic diagram of an exploded structure of the second hemispherical shell of the present invention.

FIG. 7 is a schematic diagram of the three-dimensional structure of the external controller of the present invention.

FIG. 8 is a schematic diagram of an exploded structure of the external controller of the present invention.

FIG. 9 is a schematic diagram of the usage state of the microsphere particle and the external controller of the present invention.

### DETAILED DESCRIPTION OF EMBODIMENTS

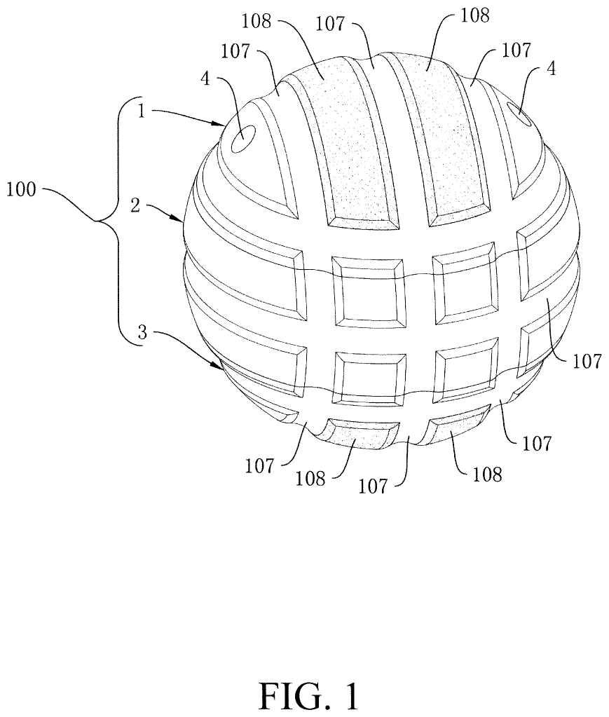

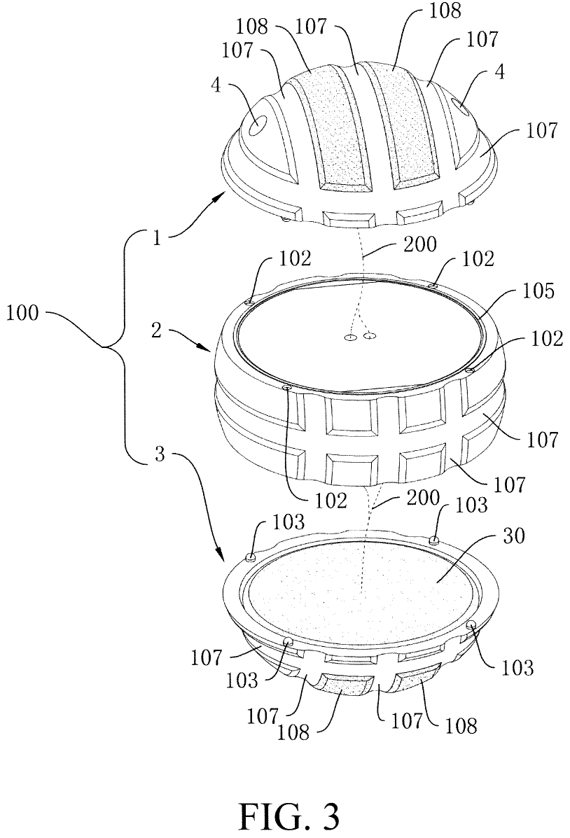

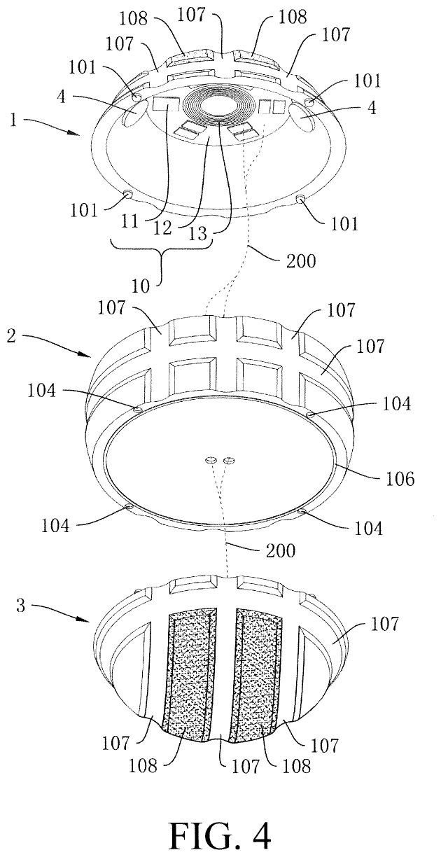

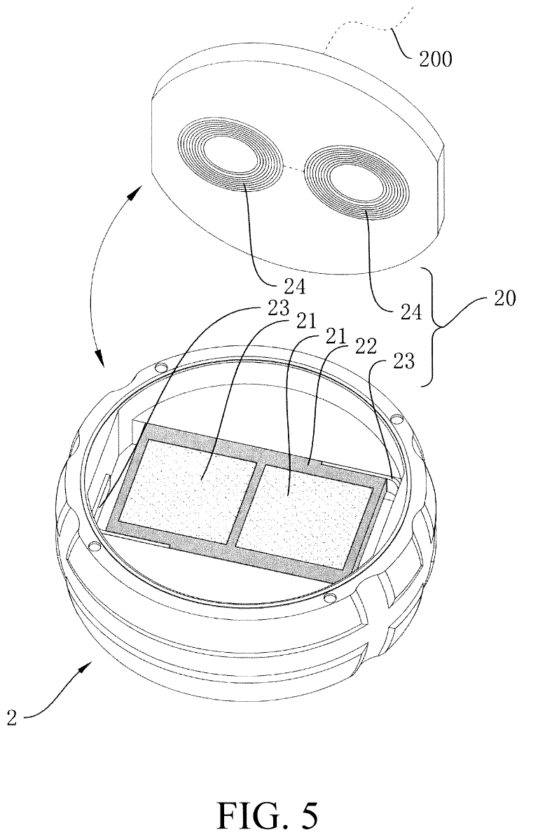

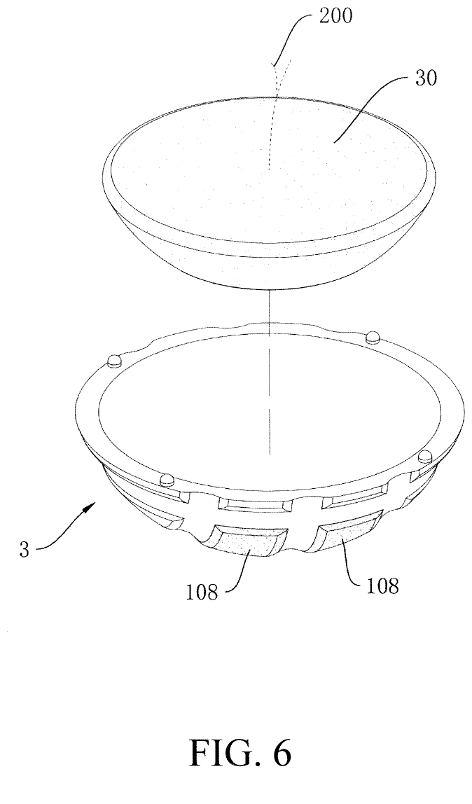

As shown in FIG. 1 and FIG. 3, the implantable nano-ultrasonic cavitation therapeutic particle of the present invention comprises a first hemispherical shell 1, a central shell 2, and a second hemispherical shell 3 produced by nanotechnology, the first hemispherical shell 1 and the second hemispherical shell 3 are connected via the central shell 2 to form a complete microsphere particle 100; as shown in FIG. 4 to FIG. 6, the central shell 2 houses a micro-ultrasonic vibration assembly 20 produced by nanotechnology, the micro-ultrasonic vibration assembly 20 comprising a sliding block 22 with an integrated permanent magnet 21, buffer springs 23 positioned at opposing ends of the sliding block 22, and a driving coil 24 configured to interact with the permanent magnet 21 to generate a magnetic driving force; the first hemispherical shell 1 incorporates a drive control assembly 10 produced by nanotechnology, the drive control assembly 10 including a circuit board 12 with an integrated wireless communication module 11, and a wireless charging receiving coil 13 electrically coupled to the circuit board 12, the driving coil 24 being electrically connected to the circuit board 12 and generating a magnetic driving force causing the sliding block 22 to reciprocate at high frequency; the second hemispherical shell 3 further incorporates a first battery 30 electrically coupled to the circuit board 12.

The working principle of the micro-ultrasonic vibration assembly 20: The first battery 30 supplies power to the driving coil 24 through the circuit board 12, current flows in the driving coil 24 to generate a magnetic field, driving the permanent magnet 21 and the sliding block 22 to reciprocate, thereby generating high-frequency vibrations. These high-frequency vibrations propagate in mediums such as gas, liquid, and solid to form ultrasonic waves.

This solution uses the setting of the wireless communication module 11 to allow users to send on and off signals to the wireless communication module 11 through wireless communication devices, so that the circuit board 12 controls the start and stop of the entire micro-ultrasonic vibration assembly 20. The wireless communication module 11 is an ultra-micro Bluetooth communication module or WiFi communication module, etc., using a wireless communication module to connect to devices such as computers, smartphones, and tablet computers, so as to control, monitor, and transmit data to the therapeutic particle of this solution.

In addition to using the aforementioned micro-ultrasonic vibration assembly 20 as an embodiment, the present invention can also be implemented by replacing it with a micro-ultrasonic transducer produced by nanotechnology, and a micro-ultrasonic vibration motor produced by nanotechnology.

In practical applications, the driving coil 24, the wireless charging receiving coil 13, and the first battery 30 are all electrically coupled to the circuit board 12 through wires 200, and the wireless communication module 11 is integrated into the circuit board 12.

In order to facilitate the assembly and positioning of the first hemispherical shell 1, the central shell 2, and the second hemispherical shell 3, and improve the convenience of assembly, as shown in FIG. 3 and FIG. 4, the first hemispherical shell 1 and the central shell 2 further include a first interlocking positioning pin 101 and a first positioning hole 102, and the second hemispherical shell 3 and the central shell 2 further include a second interlocking positioning pin 103 and a second positioning hole 104. Specifically, the first positioning pin 101 is arranged on the first hemispherical shell 1, and the first positioning hole 102 is arranged on the central shell 2. Of course, the arrangement positions of the first positioning pin 101 and the first positioning hole 102 can also be interchanged, and the interlocking and positioning function can still be achieved. Similarly, the second positioning pin 103 is arranged on the second hemispherical shell 3, and the second positioning hole 104 is arranged on the central shell 2. Of course, the arrangement positions of the second positioning pin 103 and the second positioning hole 104 can also be interchanged, and the interlocking and positioning function can still be achieved. With such a structural design, after assembly, the relative positions will not shift, improving the accuracy of positioning. In practical applications, in order to make the first hemispherical shell 1, the central shell 2, and the second hemispherical shell 3 firmly installed together and not easy to loosen, the first hemispherical shell 1 and the central shell 2 are fixed together by welding, and the second hemispherical shell 3 and the central shell 2 are also fixed together by welding.

In order to improve the sealing performance to protect the electrical components inside the microsphere particle 100, as shown in FIG. 3 and FIG. 4, the first hemispherical shell 1 and the central shell 2 are further provided with a first sealing ring 105, and the second hemispherical shell 3 and the central shell 2 are further provided with a second sealing ring 106.

In order to facilitate the observation of the operating status of the implanted nano-ultrasonic cavitation therapeutic particle inside the human body, and to detect the condition of the diseased area inside the human body, as shown in FIG. 1 and FIG. 4, the first hemispherical shell 1 or the second hemispherical shell 3 further incorporates a camera module or a sensor module 4 electrically coupled to the circuit board 12. The specific choice of camera module or sensor module 4 can be made according to the actual treatment situation. As a preferred option, in order to facilitate electrical connection with the circuit board 12, the camera module or sensor module 4 is also arranged on the first hemispherical shell 1. In use, the camera module or sensor module 4 is connected to external display screens, smartphones, tablet computers, and computers through the wireless communication module 11 on the circuit board 12 to transmit image information or sensor data to the outside and display it.

In order to prevent the microsphere particle 100 from hindering the blood flow of blood vessels after being implanted into the human body and to ensure normal blood flow, as shown in FIG. 1, the outer surface of the microsphere particle 100 is further provided with a plurality of flow guiding grooves 107 arranged in an intersecting pattern. In this way, blood stasis can be prevented after implantation, and the use is safer and more reliable.

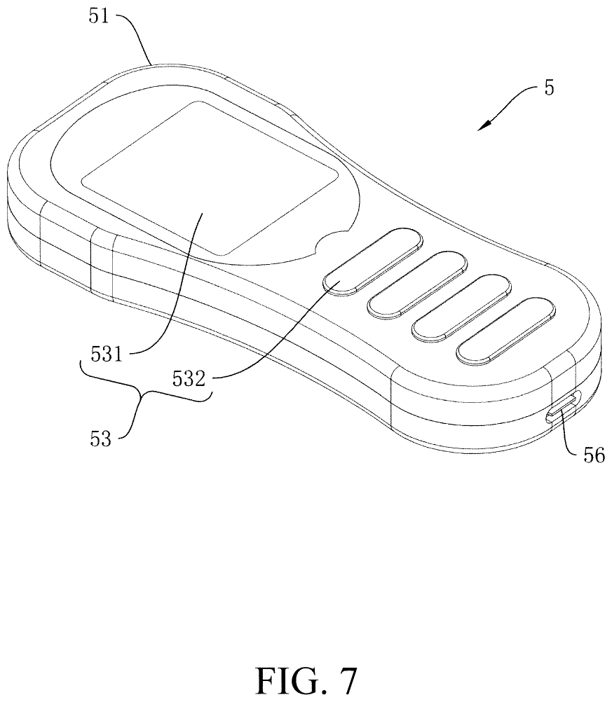

In order to facilitate the operation and monitoring of the working status of the microsphere particle 100 inside the human body, as shown in FIG. 7, the present invention also includes an external controller 5; the external controller 5 includes a housing 51, a main control circuit 52 disposed within the housing 51 and configured for bidirectional control signal communication with the wireless communication module 11, and an operation display assembly 53 mounted on the housing 51, the operation display assembly 53 being electrically coupled to the main control circuit 52. This facilitates the operation of the microsphere particle 100.

In order to further improve the structure of the operation display assembly 53, as shown in FIG. 7, the operation display assembly 53 comprises a display screen 531 and control buttons 532, the display screen 531 and control buttons 532 being each electrically coupled to the main control circuit 52. By setting the display screen 531, it is convenient to intuitively display information such as operation pages, image information, and sensor data. By setting the control buttons 532, operation is facilitated.

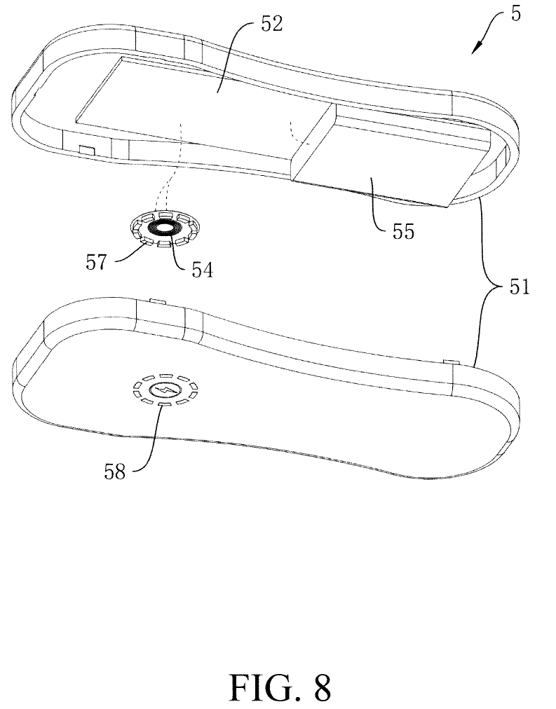

In order to facilitate the charging of the first battery 30, as shown in FIG. 4 and FIG. 8, the housing 51 further includes a wireless charging transmitting coil 54 electrically coupled to the main control circuit 52, wherein the wireless charging transmitting coil 54 is configured to transmit an electromagnetic field to the wireless charging receiving coil 13, and wherein the wireless charging receiving coil 13 is configured to convert the received electromagnetic field into a current for charging the first battery 30.

In order to improve the portability of the external controller 5, so that the external controller 5 can be carried around and used anytime and anywhere without being interfered by the distance of the wire, as shown in FIG. 7 and FIG. 8, the housing 51 further incorporates a second battery 55 electrically coupled to the main control circuit 52, and the housing 51 is further provided with a charging interface 56 electrically connected to the second battery 55.

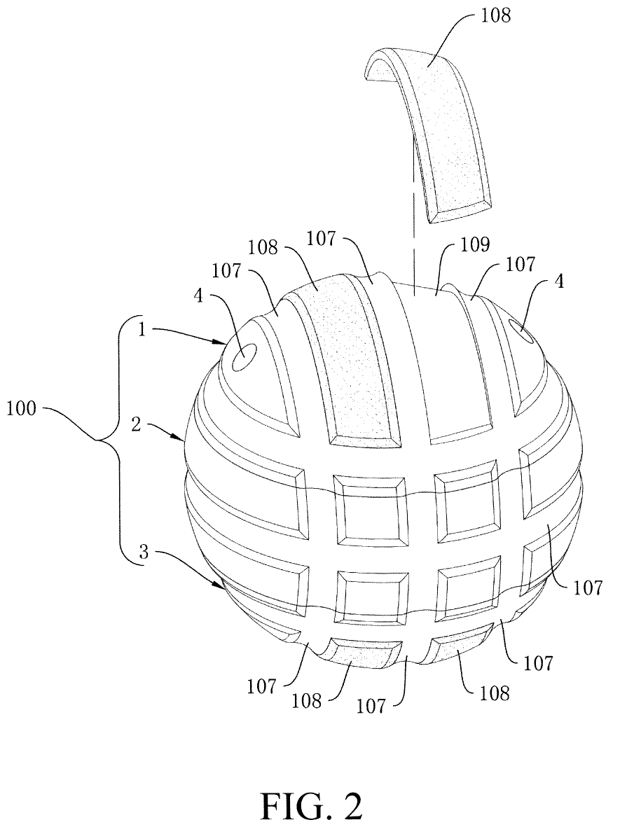

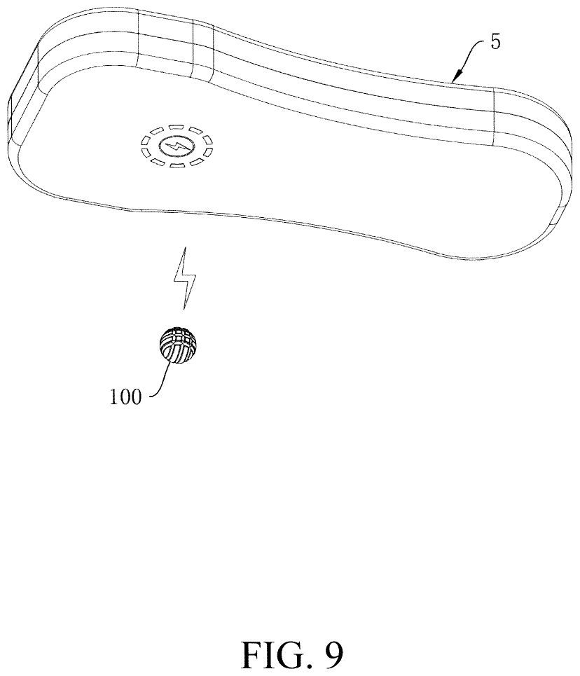

When the microsphere particle 100 is implanted into a human blood vessel for treating thrombosis, as shown in FIG. 4, FIG. 8, and FIG. 9, the housing 51 further incorporates a DC electromagnet 57 electrically coupled to the main control circuit 52, and the surface of the microsphere particle 100 is provided with a micro-magnet 108 configured for magnetic interaction with the DC electromagnet 57. The DC electromagnet 57 of the external controller 5 applies a magnetic field with opposite polarity to the micro-magnet 108 of the microsphere particle 100 to drive the microsphere particle 100 to move in the human blood vessel, so that the microsphere particle 100 can perform high-frequency ultrasonic vibration while traveling and moving in the blood vessel. Moreover, due to the assistance of high-frequency ultrasonic vibration, the resistance of the microsphere particle 100 in the blood vessel during movement is very small whether it is moving forward or backward, allowing it to travel and retreat freely without impacting or injuring the inner wall of the blood vessel. The micro-ultrasonic vibration assembly 20 is used to generate high-frequency ultrasonic vibrations, causing the blood to produce an ultrasonic cavitation effect, which causes the blood cells to move at high speed, generating micro-friction between the cells. The micro-friction can change the volume of tissue cells, reduce swelling, change the permeability of the membrane, promote the exchange of metabolic substances, change the function of cells, and improve the regeneration ability of tissue cells. At the same time, the micro-friction can consume blood clots and blood lipids in the blood. Long-term use can reduce the formation of blood clots, ensure the health of human blood vessels, and can be placed in the blood vessels for a long time without the need for cleaning and maintenance of the microsphere particle 100, and without the need for repeated removal and placement, greatly reducing the number of times of muscle trauma, and greatly reducing the suffering of the patient. Using this technical solution, it can not only be used for cleaning and maintaining human blood vessel thrombosis, but also for ultrasonic cavitation treatment of tracheal sputum in the human lung, and ultrasonic cavitation treatment of the human gastrointestinal tract. In order to facilitate the installation and positioning of the micro-magnet 108, as shown in FIG. 2, the surface of the microsphere particle 100 further includes a recessed mounting slot 109, and the micro-magnet 108 is embedded within the mounting slot 109.

In order to allow the user to quickly know the positions of the wireless charging transmitting coil 54 and the DC electromagnet 57 in the external controller 5, as shown in FIG. 8, the outer surface of the housing 51 is further provided with position indication marks 58 corresponding to the positions of the wireless charging transmitting coil 54 and the DC electromagnet 57.

In practical applications, the diameter of the microsphere particle 100 is 1 mm to 2 mm.

In practical applications, the first hemispherical shell 1, the central shell 2, and the second hemispherical shell 3 can be made of stainless steel, nickel-titanium alloy, or cobalt-chromium alloy, which have high strength and good corrosion resistance.

Compared with the prior application filed by the applicant to the China National Intellectual Property Administration under the name “A Wireless Magnetic Ultrasonic Cavitation Motion In Vivo Physical Therapy Robot Device” and the patent application number CN202010722274.7, this solution has a smaller volume, reaching the nano level, a simpler and more scientific structure, and less burden and impact on people's implantation applications, and has significant progress.

I claim:

1. An implantable nano-ultrasonic cavitation treatment particle, comprising: a first hemispherical shell (1), a middle shell (2), and a second hemispherical shell (3), wherein the first hemispherical shell (1) and the second hemispherical shell (3) are connected by the
middle shell (2) to form a complete microsphere particle (100);
the middle shell (2) includes a micro-ultrasonic vibration assembly (20), the micro-ultrasonic vibration assembly (20) comprising a sliding block (22) with a permanent magnet (21), springs (23) arranged at both ends of the sliding block (22) for buffering and rebounding, and a driving coil (24) that cooperates with the permanent magnet (21) to generate a magnetic driving force;
the first hemispherical shell (1) includes a driving control assembly (10), the driving control assembly (10) comprising a circuit board (12) with a wireless communication module (11) and a wireless charging receiving coil (13) electrically connected to the circuit board (12), the driving coil (24) is electrically connected to the circuit board (12) and generates a driving magnetic force to cause the sliding block (22) to perform high-frequency reciprocating motion;
the second hemispherical shell (3) includes a first battery (30) electrically connected to the circuit board (12).

2. The implantable nano-ultrasonic cavitation treatment particle according to claim 1, wherein the first hemispherical shell (1) and the middle shell (2) further include a first positioning pin (101) and a first positioning hole (102) that are nested together, and the second hemispherical shell (3) and the middle shell (2) further include a second positioning pin (103) and a second positioning hole (104) that are nested together.

3. The implantable nano-ultrasonic cavitation treatment particle according to claim 1, wherein the first hemispherical shell (1) and the middle shell (2) further include a first sealing ring (105), and the second hemispherical shell (3) and the middle shell (2) further include a second sealing ring (106).

4. The implantable nano-ultrasonic cavitation treatment particle according to claim 1, wherein the first hemispherical shell (1) or the second hemispherical shell (3) further includes a camera module or a sensor module (4) electrically connected to the circuit board (12).

5. The implantable nano-ultrasonic cavitation treatment particle according to claim 1, wherein the outer surface of the microsphere particle (100) further includes a plurality of flow guiding grooves (107) arranged in a crisscross pattern.

6. The implantable nano-ultrasonic cavitation treatment particle according to claim 1, further comprising an external controller (5);
wherein the external controller (5) comprises a housing (51), a main control circuit (52) arranged in the housing (51) and configured to transmit and receive control signals with the wireless communication module (11), and an operation display assembly (53) arranged on the housing (51), the operation display assembly (53) being electrically connected to the main control circuit (52).

7. The implantable nano-ultrasonic cavitation treatment particle according to claim 6, wherein the operation display assembly (53) comprises a display screen (531) and operation buttons (532), the display screen (531) and operation buttons (532) being respectively electrically connected to the main control circuit (52).

8. The implantable nano-ultrasonic cavitation treatment particle according to claim 6, wherein the housing (51) further includes a wireless charging transmitting coil (54) electrically connected to the main control circuit (52), the wireless charging transmitting coil (54) configured to transmit an electromagnetic field to the wireless charging receiving coil (13), and the wireless charging receiving coil (13) configured to convert the received electromagnetic field into current to charge the first battery (30).

9. The implantable nano-ultrasonic cavitation treatment particle according to claim 6, wherein the housing (51) further includes a second battery (55) electrically connected to the main control circuit (52), and the housing (51) further includes a charging interface (56) electrically connected to the second battery (55).

10. The implantable nano-ultrasonic cavitation therapeutic particle according to claim 6, wherein the housing (51) further incorporates a DC electromagnet (57) electrically coupled to the main control circuit (52), and wherein the surface of the microsphere particle (100) is provided with a micro-magnet (108) configured for magnetic interaction with the DC electromagnet (57); the surface of the microsphere particle (100) further includes a recessed mounting slot (109), and the micro-magnet (108) is embedded within the mounting slot (109).
